(19) United States
(12) Patent Application Publication
(10) Pub. No.: US 2008/0009756 A1
(43) Pub. Date: Jan. 10, 2008

(54) Method and apparatus for assessing vascular conditions

(75) Inventor: Hanan Keren, Kfar-Saba (IL)

Correspondence Address: Martin D. Moynihan;PRTSI, Inc., P.O. Box 16446, Arlington, VA, 22215, US

(73) Assignee: New Leaf Capital Ltd., Road Town (VG)

(21) Appl. No.: 11/822,645

(22) Filed: Jul. 9, 2007

Related U.S. Application Data
(60) Provisional application No. 60/819,402, filed on Jul. 10, 2006.

(51) Int. Cl. A61B 5/026; A61B 5/0402; A61B 5/103

(52) U.S. Cl. 600/504; 600/509; 600/595

(57) ABSTRACT

A method suitable for assessing the vascular condition of a subject is disclosed. The method comprises: measuring the transit time TT₁ of a blood wavefront from the subject's heart to a distal point in a limb of the subject, and measuring the transit time TT₂ of a blood wavefront from an intermediate point in the limb of the subject to the distal point of the subject's limb. The method further comprises computing the ratio TT₂/TT₁ and determining the likelihood that a small vessel disease condition exists when the ratio TT₂/TT₁ is above a preselected threshold.

### RELATED APPLICATIONS

This application claims the benefit of U.S. Provisional Patent Application No. 60/819,402 filed Jul. 10, 2006, the contents of which are hereby incorporated in its entirety.

### FIELD AND BACKGROUND OF THE INVENTION

The present application relates to a method and apparatus for assessing the vascular condition of a subject, and particularly, but not exclusively, for determining the likelihood that a small vessel disease condition exists in the subject.

Small vessel disease (SVD) conditions generally accompany common microvascular complications of diabetes. This condition usually involves the small arteries at the lower extremities of the body. Affected individuals are therefore at risk for decreased blood flow to the legs and feet, which often leads to pain, numbness, functional impairments, tissue loss, gangrene and/or amputation. Such a condition also is often a marker for generalized arteriosclerosis, and therefore serves notice that the individual is at increased risk of myocardial infarct, stroke, and possibly death.

Diagnosing this condition is very difficult for several reasons. For example, conventional angioplasty requires the injection of a dye, which may be harmful to the kidneys of a diabetic. Moreover, such techniques, as well as Doppler techniques for measuring blood flow, generally require specialized equipment which is not commonly available. Accordingly, at the present time the usual technique for detecting SVD conditions is by Background Diabetes Retinopathy (BDR), which also requires very specialized equipment, as well as being highly intrusive of the patient. As a result, many cases of SVD conditions go undetected, thereby jeopardizing the health of the subject.

U.S. Pat. No. 6,676,608 to Kern discloses a method and apparatus for monitoring the cardiovascular condition of a subject, particularly the degree of arteriosclerosis. As described by Keren, this is done by detecting an ECG signal of the individual's heart; detecting a blood wavefront in a peripheral artery of the individual; and measuring the time lag between a predetermined reference point in the detected blood front wave and a predetermined reference in the ECG signal such as to provide an indication of the presence of arteriosclerosis in the subject. Further details of the method and apparatus are available in U.S. Pat. No. 6,676,608, the contents of which are incorporated herein by reference.

### SUMMARY OF THE INVENTION

An object of the present invention is to provide a method and apparatus for assessing the vascular condition of a subject, e.g., for determining the likelihood that an SVD condition exists. The technique is preferably based on the technique described in U.S. Pat. No. 6,676,608.

According to one aspect of the present invention there is provided a method of assessing the vascular condition of a subject. The method comprises measuring transit times of a blood wavefront along at least two segments in the body of the subject, using the transit times for calculating a predefined vascular condition index, and assessing the vascular condition based on a value of the vascular condition index.

According to further features in preferred embodiments of the invention described below, at least one segment is defined between the heart of the subject and a location in a limb of the subject. According to still further features in the described preferred embodiments at least one segment is defined between two locations in a limb of the subject.

Thus, in various exemplary embodiments of the invention the method begins by measuring the transit time TT₁ of a blood wavefront from the heart to a distal point in the limb (e.g., a toe), and continues by measuring the transit time TT₂ of a blood wavefront from an intermediate point in the limb (e.g., a point in the ankle region) to the distal point. In various exemplary embodiments of the invention the predefined vascular condition index is the ratio of TT₂/TT₁. In these embodiments, the method preferably calculates the ratio of TT₂/TT₁ and determines the likelihood that an SVD condition exists when the ratio is above a preselected threshold.

Thus, as described in the aboveited U.S. Pat. No. 6,676,608, there is a correlation between the cardiovascular condition of an individual and the time lag experienced by the blood wavefront in traveling from the individual's heart to a peripheral artery. As described in that patent, in healthy individuals, this time lag varies approximately linearly with age, decreasing about 1 ms for each year.

Preferred embodiments of the present invention are based on the appreciation that this time lag measurement can also be used for vasculature condition assessment, particularly for determining whether or not an SVD condition exists. The ratio TT₂/TT₁ in a healthy individual is usually less than about a fifth. Thus, when this ratio is found to exceed the preselected threshold, there is a likelihood that an SVD condition exists in the limb of the subject, between the mentioned intermediate and distal points.

As indicated above, in the described preferred embodiments of the invention, the distal point in the subject's limb is the toe of a foot of the subject and the intermediate point in the subject's limb is a point in the ankle region of the foot of the subject. As further indicated above, the preselected threshold is equivalent to about 20 percents (namely a ratio of about fifth).

In the preferred embodiments of the invention described below, the transit times TT₁ and TT₂ are measured by using a predetermined reference point in the output signals of blood wavefront sensors at the respective intermediate and distal points of the subject's limb as a time marker for marking the arrival times of the blood wavefront at the intermediate point and distal point, respectively, in the limb of the subject.

In one described preferred embodiment the transit time TT₁ is measured by utilizing a predetermined reference point in the subject's ECG signal as a time marker for marking the starting time of the blood wavefront from the subject's heart.

In a second described embodiment, the transit time TT₁ is measured by utilizing a predetermined reference point in the output signal of a blood wavefront sensor located at the heart region of the subject as a time market for marking the starting time of the blood wavefront from the subject's heart.

The invention also provides apparatus for assessing the vascular condition of a subject according to the above-described method. The apparatus preferably comprises sensors adapted for measuring transit times of a blood wavefront along at least two segments in the body of the subject; a data processor for calculating a predefined vascular condition index using the transit times; and a display device for displaying a value of the vascular condition index to thereby provide an indication of the vascular condition, as described herein.

The invention further provides a method of characterizing vascular conditions. The method preferably calculates the predefined vascular condition index such as to characterize the vascular condition.

Further features and advantages of the invention will be apparent from the description below.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a diagram illustrating one form of apparatus for assessing the vascular condition of a subject in accordance with the present invention;

FIGS. 2a-c are fragmentary views of FIG. 1 more particularly illustrating the sensors for sensing the blood wavefronts;

FIGS. 3a and 3b illustrates examples of wave forms produced with the apparatus of FIG. 1, indicating the lack of an SVD condition (FIG. 3a) and the presence of an SVD condition (FIG. 3b), respectively;

FIG. 4 is a block diagram illustrating another apparatus constructed in accordance with the present invention for assessing the vascular condition of a subject; and

FIG. 5 is a block diagram illustrating another apparatus constructed in accordance with the present invention for assessing the vascular condition of a subject.

### DESCRIPTION OF THE PREFERRED EMBODIMENTS

The present embodiments comprise a method and apparatus which can be used for assessing vascular conditions. Specifically, the present embodiments can be used to determine the likelihood that a small vessel disease condition exists in the subject The principles and operation of a method and apparatus according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

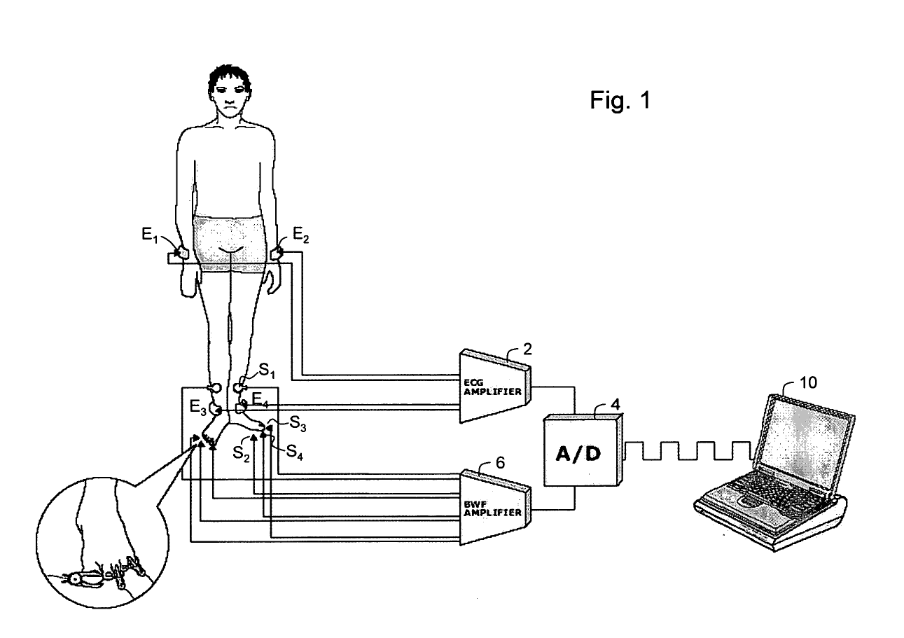

Referring now to the drawings, FIG. 1 illustrates one form of apparatus constructed in accordance with the present invention for assessing the vascular condition of a subject, particularly, but not exclusively, for indicating the likelihood that a small vessel disease (SVD) condition exists in the subject.

Generally, this is done by measuring transit times of a blood wavefront along two or more segments in the body of the subject, using the transit times for calculating a predefined vascular condition index, and assessing the vascular condition based on a value of the vascular condition index. The segments in the body are typically blood flow paths along the vasculature. In various exemplary embodiments of the invention at least one of the segments is defined between the heart of the subject and a location in a limb of the subject. Another segment is preferably defined between two locations in the limb. It was found by the Inventor of the present invention that it is particularly useful to define one segment between the heart of the subject and a distal point in the limb (e.g., a toe), and another segment between an intermediate point in the same limb (e.g., a point in the ankle region) to the distal point.

Thus, the transit times of the present embodiments preferably comprise a firth transit time TT₁ of a blood wavefront from the heart to the distal point, and a second transit time TT₂ of a blood wavefront from the intermediate point to the distal point.

In various exemplary embodiments of the invention the predefined vascular condition index is the ratio of TT₂/TT₁. But this need not necessarily be the case, since, for some applications, it may not be necessary to define the index as TT₂/TT. Also contemplated are indices defined as (TT₁−TT₂)/TT₁, (TT₁−TT₂)/(TT₁+TT₂), TT₁−TT₂ and the like.

In any event, once the index is calculated the method assesses the vascular condition based on the value of the index. For example, the method can determine the likelihood that an SVD condition exists by comparing the value of the index to a preselected threshold. It is appreciated that both the value of the preselected threshold and the criterion for determining the likelihood that an SVD condition exists depend on the definition of the index. For example, in the preferred embodiment in which the index is the ratio TT₂/TT₁, the method determines the likelihood that an SVD condition exists when the ratio is above the preselected threshold. In this embodiment, the preselected threshold can be equivalent to 20%.

As used herein the term “about” refers to ±10%.

In other embodiments (e.g., when the index is defined as (TT₁−TT₂)/T₁, (TT₁−TT₂)/(TT₁+TT₂), TT₁−TT₂), the method determines the likelihood that an SVD condition exists when the ratio is below the threshold, and the value of the threshold is selected accordingly.

In the apparatus illustrated in FIG. 1, the subject's ECG signal is used as a time marker for marking the starting time of the blood wavefront from the subject's heart. Accordingly, the apparatus illustrated in FIG. 1 includes a plurality of ECG electrodes E₁-E₄ connected to an ECG amplifier 2 for sensing the subject's ECG signal. Amplifier 2 produces an output to an analogue-to-digital converter 4 so as to enable digital processing of the ECG signal, as will be described more particularly below.

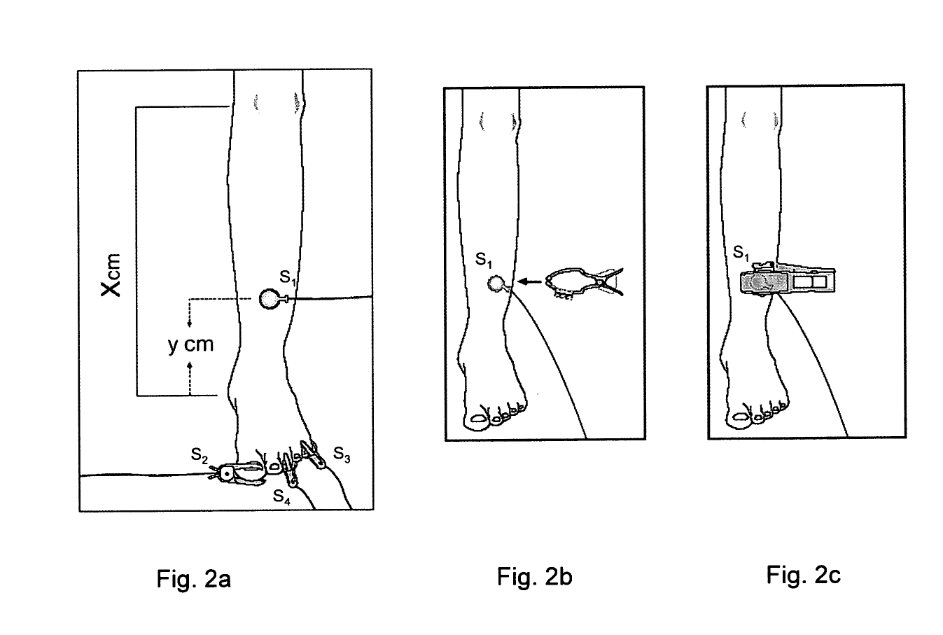

The apparatus illustrated in FIG. 1 further includes a plurality of blood wavefront sensors S₁-S₄ on one leg of the subject, and a similar plurality of sensors on the other leg of the subject. Sensors S₁-S₄ are more particularly illustrated in FIGS. 2a-c, wherein it will be seen that sensor S₁ is applied to the ankle region of the respective foot; sensor S₂ is applied to the big toe of the respective foot; sensor S₃ is applied to the little toe of the respective foot; and sensor S₄ is applied to the middle toe of the respective foot.

Sensors S₁-S₄ may be any known type of blood wavefront sensor. It senses a blood wavefront arriving at the respective point of the subject's leg and produces an output signal corresponding to the sensed blood wavefront. For example, sensors S₁-S₄ may be of the infrared-type oximeter measuring the oxygen saturation of the blood, such as are known for measuring pulse rate and plethysmographic pulse waves, as described in the above-cited U.S. Pat. No. 6,676,608. Sensors S₁-S₄ may also be of the movement-detection or vibration-detection type, which detects the actual movement of the skin caused by the pulsatile blood flow, such as those supplied by Nexense Ltd., of Yavne, Israel, and described in International Patent Application PCT/IL2004/000138, Publication No. WO2004/072658, published Aug. 26, 2004.

As shown in FIG. 1, the outputs of the blood wavefront sensors S₁-S₄ of both legs of the subject are amplified in an amplifier 6 before being applied to the A/D converter 4 for further processing, as described below.

FIGS. 2a-c illustrate a technique for connecting the sensors to amplifier 6. FIG. 2a also illustrates a preferred location for the ankle sensor S₁, but as will be described more particularly below, this location is not critical. As shown in FIG. 2a, the ankle sensor S₁ is located a distance Y from the heel of the subject's leg, which distance Y is about ⅓ the distance X, namely the distance of the subject's knee from the heel. However, as indicated above, this precise location of the ankle sensor S₁ is not critical in most cases since if an SVD condition exists, it usually exists, or is much more severe, in the foot of the subject rather than in the general ankle region; therefore the transit time of the blood wavefront from the knee to the ankle sensor S₁ is usually very small as compared to the transit time of the blood wavefront from the ankle sensor S₁ to the respective toe sensors S₂-S₄.

By using a plurality of toe sensors S₂-S₄, separate tests may be made, as described more particularly below, to provide a better indication of the location and the severity of the SVD condition, if one is found to exist.

As shown in FIG. 1, the ECG signal from electrodes E₁-E₄, after being amplified in amplifier 2 and converted to digital form in A/D converter 4, are fed to a data processor, generally designated 10, to be used as a time marker for marking the starting time of the blood wavefront from the subject's heart. In addition, the outputs from the ankle sensors S₁ and toe sensors S₂-S₄, for the respective leg of the subject, after being amplified in amplifier 6 and converted to digital form in A/D converter 4, are also fed to data processor 10.

Data processor 10 utilizes the ECG signals and blood wavefront signals for producing a measurement of the transit time TT₁ of the blood wavefront from the subject's heart to the respective toe of the subject, and also for producing a measurement of the transit time TT₂ of the blood wavefront from the subject's ankle to the respective toe of the subject. Data processor 10 also computes the ratio of TT₂/TT₁. If this ratio is found to be less than 20 percent, this would indicate the likelihood that an SVD condition does not exist; whereas if the ratio TT₂/TT₁ is found to be above 20 percent, this would indicate the likelihood that an SVD condition does indeed exist.

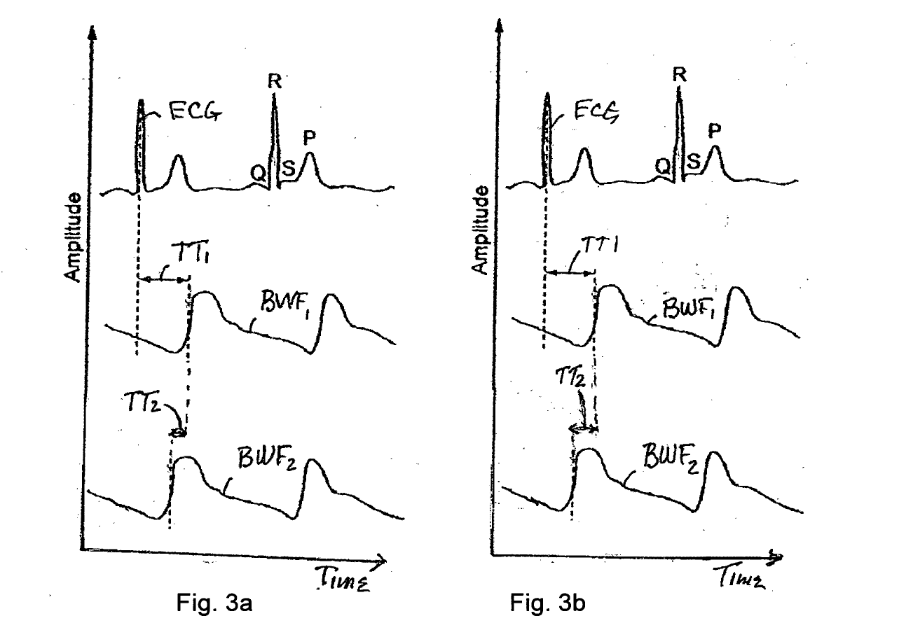

The foregoing is more particularly illustrated in FIGS. 3a and 3b. In FIG. 3a, the blood wavefront signal BWF₁ indicates the transit time (TT₁) of the blood wavefront from the heart to the respective toe as being about 200 ms. In FIG. 3a, the blood wavefront signal BWF₂ indicates the transit time (TT₂) of the BWF wave from the ankle sensor S₁ to the respective toe sensor S₂-S₄ is shown as being about 25 ms. The ratio TT₂/TT₁ would therefore be about 12.5 percent, which would indicate that no SVD condition exists.

On the other hand, FIG. 3b illustrates a situation wherein the likelihood an SVD condition is found to exist, since in this case the transit time TT₁ of the blood wavefront signal BWF₁ from the heart to the respective toe is about 250 ms, whereas the transit time TT₂ of the blood wavefront BWF₂ from the ankle sensor S₁ to the respective toe is about 75 ms, such that the ratio TT₂/TT₁ is approximately 30 percent. This would indicate the likelihood of an SVD condition exists in the region between the ankle sensor S₁ and the respective toe sensors S₂-S₄.

As indicated earlier, a separate test as described above can be performed with each of the toe sensors S₂-S₄ in order to provide a better indication of the location, and the severity, of the SVD condition in the respective foot of the subject.

FIG. 1 illustrates the data processor 10 as being a general purpose computer programmed for performing the above-described measurements and computations. It will be appreciated that the data processor can be a dedicated computer dedicated for this particular processing operation. It will also appreciated that data processor 10 could be at a remote location in communication with the apparatus illustrated in FIG. 1 via the Internet or other communication network.

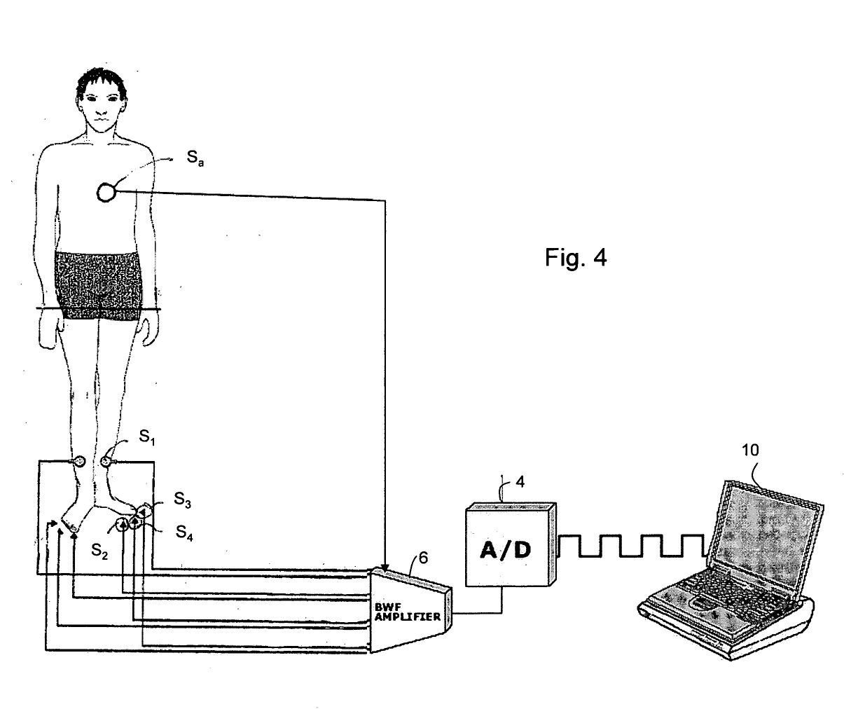

FIG. 4 illustrates apparatus similar to that of FIG. 1, except that, instead of using the subject's ECG signal as a time marker for marking the starting time of the blood wavefront from the subject's heart, there is used instead a movement-type or vibration-type sensor located at the subject's heart region for directly-detecting the heart-produced movements of the subject's skin. Such a sensor is shown at Sa in FIG. 4 as applied in direct contact with the subject's skin in the heart region of the subject. As indicated above, an example of such a movement-type or vibration-type sensor that may be used is one supplied by Nexense Ltd. of Yavne, Israel, as described for example in the above-cited International Patent Application Publication.

It will also be appreciated that the ankle sensor S₁ and/or toe S₂-S₄, could also be of the above movement-type or vibration-type sensor.

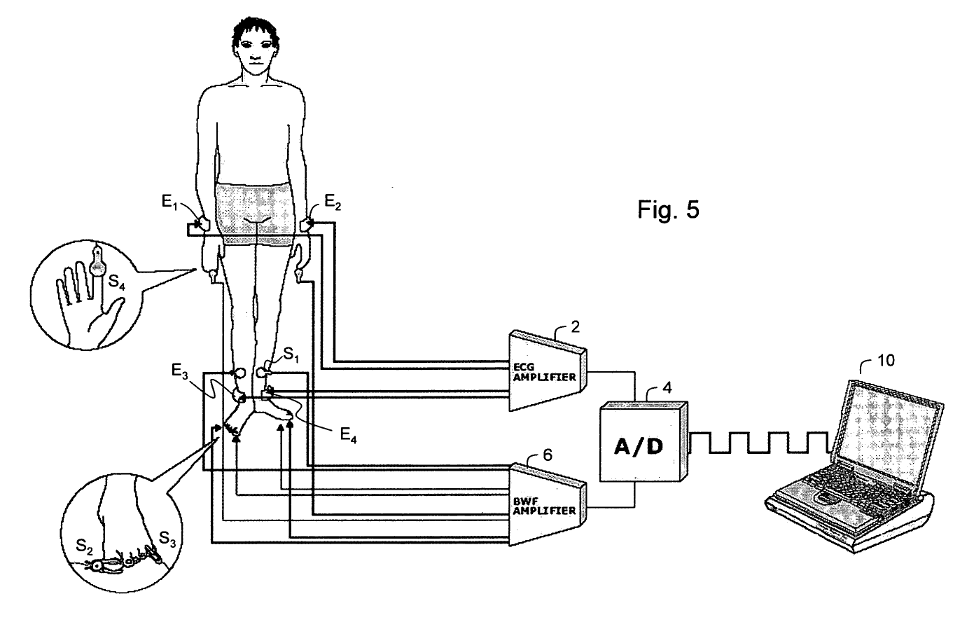

FIG. 5 illustrates apparatus similar to that of FIG. 1, except that sensor S₄ is applied to the finger (the index finger in the present example) instead of the middle toe as in FIG. 1.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method of assessing the vascular condition of a subject, comprising measuring transit times of a blood wavefront along at least two segments in the body of the subject, using said transit times for calculating a predefined vascular condition index, and assessing the vascular condition based on a value of said vascular condition index.

2. The method of claim 1, wherein at least one segment is defined between the heart of the subject and a location in a limb of the subject.

3. The method of claim 1, wherein at least one segment is defined between two locations in a limb of the subject.

4. The method of claim 2, wherein said at least two segments comprise a first segment defined between the heart and a distal point in said limb, and a second segment defined between an intermediate point in said limb and said distal point.

5. The method of claim 1, wherein said assessing the vascular condition comprises determining the likelihood that a small vessel disease condition exists.

6. The method of claim 4, wherein said vascular condition index comprises the ratio TT₂/TT₁, said TT₁ being a first transit time from the heart to said distal point and said TT₂ is a second transit time of blood wavefront from said intermediate point to said distal point.

7. The method of claim 6, wherein said assessing the vascular condition comprises if said ratio is above a preselected threshold then determining the likelihood that a small vessel disease condition exists.

8. The method of claim 4, wherein said distal point in the subject's limb is a toe of a foot of the subject; and said intermediate point in the subject's limb is a point in the ankle region of said foot of the subject.

9. The method of claim 7, wherein said preselected threshold is equivalent to about 20 percents.

10. The method of claim 4, wherein said transit times are measured by using a predetermined reference point in the output signals of blood wavefront sensors at the respective intermediate and distal points of the subject's limb as a time marker for marking the arrival times of the blood wavefront at said intermediate point and distal point, respectively, in said limb of the subject.

11. The method of claim 6, wherein said TT₁ is measured by utilizing a predetermined reference point in the subject's ECG signal as a time marker for marking the starting time of the blood wavefront from the subject's heart.

12. The method of claim 6, wherein said TT₁ and TT₂ are measured by utilizing a predetermined reference point in the output signal of a blood wavefront sensor located at the heart region of the subject as a time marker for marking the starting time of the blood wavefront from the subject's heart.

13. Apparatus for assessing the vascular condition of a subject, comprising:
sensors adapted for measuring transit times of a blood wavefront along at least two segments in the body of the subject;
a data processor for calculating a predefined vascular condition index using said transit times; and
a display device for displaying a value of said vascular condition index to thereby provide an indication of the vascular condition.

14. The apparatus of claim 13, wherein at least one of said sensors is adapted for sensing the arrival time of the blood wavefront at a location in limb of the subject.

15. The apparatus of claim 13, wherein said data processor is configured for signaling said display device to provide an indication of the likelihood that a small vessel disease condition exists.

16. The apparatus of claim 14, wherein said at least two segments comprise a first segment defined between the heart and a distal point in said limb, and a second segment defined between an intermediate point in said limb and said distal point.

17. The apparatus of claim 16, wherein said vascular condition index comprises the ratio TT₂/TT₁, said TT₁ being a first transit time from the heart to said distal point and said TT₂ is a second transit time of blood wavefront from said intermediate point to said distal point.

18. The apparatus of claim 17, wherein said data processor is configured to compare said ratio with a preselected threshold, and to signal said display device to provide an indication of the likelihood that a small vessel disease condition exists if said ratio is above said preselected threshold.

19. The apparatus of claim 18, wherein said preselected threshold is equivalent to about 20 percents.

20. The apparatus of claim 12, wherein said sensors comprise a toe sensor for sensing the arrival time of the blood wavefront at the subject's toe for determining said TT₁, and an ankle sensor for sensing the arrival time of the blood wavefront at the subject's ankle for determining said TT₂.

21. The apparatus of claim 12, wherein said sensors comprise ECG signal sensors for sensing the ECG signal of the subject, wherein said signal is used by said data processor as a time marker for marking the starting time of the blood wavefront from the subject's heart for determining said TT₁.

22. The apparatus of claim 12, wherein said sensors comprise a movement sensor for directly sensing movement in the region of the subject's heart and for producing an output signal corresponding to such movement, said output signal being used by said data processor as a time marker for marking the starting time of the blood wavefront from the subject's heart for determining said TT₁.

23. A method of characterizing vascular of a subject, comprising calculating a predefined vascular condition index based on transit times of a blood wavefront along at least two segments in the body of the subject thereby characterizing the vascular condition.

24. The method of claim 23, wherein at least one segment is defined between the heart of the subject and a location in a limb of the subject.

25. The method of claim 23, wherein at least one segment is defined between two locations in a limb of the subject.

26. The method of claim 24, wherein said at least two segments comprise a first segment defined between the heart and a distal point in said limb, and a second segment defined between an intermediate point in said limb and said distal point.

27. The method of claim 26, wherein said vascular condition index comprises the ratio TT₂/TT₁, said TT₁ being a first transit time from the heart to said distal point and said TT₂ is a second transit time of blood wavefront from said intermediate point to said distal point.

28. The method of claim 27, wherein said characterization comprises the likelihood that a small vessel disease condition exists.

29. The method of claim 26, wherein said distal point in the subject's limb is a toe of a foot of the subject; and said intermediate point in the subject's limb is a point in the ankle region of said foot of the subject.
